(19) United States
(12) Patent Application Publication
(10) Pub. No.: US 2015/0125486 A1
(43) Pub. Date: May 7, 2015

(54) ADJUVANTED FORMULATIONS OF PEDIATRIC ANTIGENS

(71) Applicant: Novartis AG, Basel (CH)

(72) Inventors: Simone Bufali, Siena (IT); Barbara Baudner, Siena (IT); Derek O'Hagan, Winchester, MA (US); Manmohan Singh, Cary, NC (US)

(21) Appl. No.: 14/381,438

(22) PCT Filed: Mar. 8, 2013
(86) PCT No.: PCT/EP2013/054674

§ 371 Date: Aug. 27, 2014

Related U.S. Application Data
(60) Provisional application No. 61/608,409, filed on Mar. 8, 2012.
(60) Provisional application No. 61/697,745, filed on Sep. 6, 2012.

(51) Int. Cl. A61K 39/39; C12N 7/00; A61K 39/095; A61K 39/09; A61K 39/00; A61K 39/13

(52) U.S. Cl. CPC A61K 39/39; A61K 39/0018; A61K 39/13; A61K 39/095; A61K 39/092; C12N 7/00; A61K 2039/55572; A61K 2039/55505; C12N 2770/32634
USPC 424/201.1; 424/203.1

(57) ABSTRACT

An immunogenic composition comprising a diphtheria toxoid, a tetanus toxoid, a pertussis toxoid, an aluminium salt adjuvant, and a TLR4 agonist. Preferably, the TLR4 agonist and/or at least one of the toxoids is/are adsorbed to the aluminium salt adjuvant.

This application claims the benefit of US provisional applications 61/608,409 (filed Mar. 8th, 2012) and 61/697,745 (filed Sep. 6, 2012), the complete contents of both of which are hereby incorporated herein by reference for all purposes.

### TECHNICAL FIELD

This invention is in the field of combination vaccines i.e. vaccines containing mixed immunogens from more than one pathogen, such that administration of the vaccine can simultaneously immunize a subject against more than one pathogen.

### BACKGROUND ART

Vaccines containing antigens from more than one pathogenic organism within a single dose are known as “multivalent” or “combination” vaccines. Various combination vaccines have been approved for human use, including trivalent vaccines for protecting against diphtheria, tetanus and pertussis (“DTP” vaccines) or measles, mumps and rubella (“MMR” vaccines). These vaccines offer patients the advantage of receiving a reduced number of injections, which can lead to the clinical advantage of increased compliance (e.g. see chapter 29 of ref. 1), particularly in pediatric patients.

Current combination vaccines can include relatively high amounts of aluminium salts as adjuvants which causes concern to some patient pressure groups despite empirical safety studies [2,3]. For instance, the levels in known combination vaccines are as follows (see also Table A below):

| Trade name | Antigens | Al⁺⁺⁺ content per unit dose | |
|---|---|---|---|
| Pediacel | D-T-Pa-Hib-IPV | 0.33 | mg |
| Pediarix | D-T-Pa-HBV-IPV | ≦0.85 | mg |
| Pentacel | D-T-Pa-Hib-IPV | 0.33 | mg |
| Tritanrix-HepB | D-T-Pw-HBV | 0.63 | mg |
| Quinvaxem | D-T-Pw-Hib-HBV | 0.3 | mg |
| Hexavac | D-T-Pa-IPV-Hib-HBV | 0.3 | mg |
| Boostrix (USA) | D-T-Pa | ≦0.39 | mg |

A vaccine with lower levels of aluminium would be helpful for some patient groups, and it is an object of the present invention to provide such vaccines, ideally without loss of vaccine potency.

Another drawback with current vaccines is that they require relatively high amounts of antigen, whereas various documents show that protective effects might be achieved with lower amounts of antigen e.g. reference 4 shows that the amount of Hib antigen can be halved in a D-T-Pw-Hib vaccine without loss of immunological response, and reference 5 argues that a reduced IPV dose can be used while maintaining an adequate level of protection against polio. It is an object of the present invention to provide further vaccines with reduced amounts of antigen, ideally without loss of immunoprotective effect.

### SUMMARY OF THE INVENTION

In general, the invention provides an immunogenic composition comprising a diphtheria toxoid, a tetanus toxoid, a pertussis toxoid, an aluminium salt adjuvant, and a TLR4 agonist. Preferably, the TLR4 agonist and/or at least one of the toxoids is/are adsorbed to the aluminium salt adjuvant.

The invention thus provides a variety of combination vaccine compositions as well as methods for their manufacture. By including a TLR4 agonist it is possible for the compositions to have a relatively low amount of antigen and/or a relatively low amount of aluminium, while nevertheless having immunogenicity which is comparable to combination vaccines with a relatively high amount of antigen and/or a relatively high amount of aluminium.

Thus in a first embodiment the immunogenic composition has a concentration of Al⁺⁺⁺ of less than 0.4 mg/ml. Where the immunogenic composition is in a unit dose form for administration to a patient, the amount of Al⁺⁺⁺ in the unit dose can be less than 0.2 mg.

In a second embodiment the composition has a low dose of each of a diphtheria toxoid, a tetanus toxoid, and a pertussis toxoid.

In a third embodiment the composition has (a) a low dose of each of a diphtheria toxoid, a tetanus toxoid, and a pertussis toxoid and (b) a concentration of Al⁺⁺⁺ of less than 0.4 mg/ml. Where the composition is in a unit dose form for administration to a patient, it can comprise less than 0.2 mg Al⁺⁺⁺ per unit dose.

Compositions of the invention can include antigens in addition to diphtheria toxoid, tetanus toxoid, and pertussis toxoid e.g. they can include Hib capsular saccharide (ideally conjugated), HBsAg, IPV, meningococcal capsular saccharide (ideally conjugated), etc.

A further aspect of the invention is an immunisation schedule for an infant in which only one or two DTaP-containing compositions of the invention are administered. Thus the invention provides a method for immunising an infant against at least diphtheria, tetanus and pertussis (whooping cough), comprising administering to the infant no more than two doses of a combination vaccine of the invention.

### Diphtheria Toxoid

Diphtheria is caused by Corynebacterium diphtheriae, a Gram-positive non-sporing aerobic bacterium. This organism expresses a prophage-encoded ADP-ribosylating exotoxin (‘diphtheria toxin’), which can be treated (e.g. using formaldehyde) to give a toxoid that is no longer toxic but that remains antigenic and is able to stimulate the production of specific anti-toxin antibodies after injection. Diphtheria toxoids are disclosed in more detail in chapter 13 of reference 1. Preferred diphtheria toxoids are those prepared by formaldehyde treatment. The diphtheria toxoid can be obtained by growing C. diphtheriae in growth medium (e.g. Fenton medium, or Linggoud & Fenton medium), which may be supplemented with bovine extract, followed by formaldehyde treatment, ultrafiltration and precipitation. The toxoided material may then be treated by a process comprising sterile filtration and/or dialysis.

Quantities of diphtheria toxoid can be expressed in international units (IU). For example, the NIB SC [6] supplies the ‘Diphtheria Toxoid Adsorbed Third International Standard 1999’ [7,8], which contains 160 IU per ampoule. As an alternative to the IU system, the ‘LI’ unit (“flocculating units”, the “limes flocculating dose”, or the “limit of flocculation”) is defined as the amount of toxoid which, when mixed with one International Unit of antitoxin, produces an optimally flocculating mixture [9]. For example, the NIBSC supplies ‘Diphtheria Toxoid, Plain’ [10], which contains 300 Lf per ampoule and ‘The 1st International Reference Reagent For Diphtheria Toxoid For Flocculation Test’ [11] which contains 900 Lf per ampoule. The concentration of diphtheria toxin in a composition can readily be determined using a flocculation assay by comparison with a reference material calibrated against such reference reagents. The conversion between IU and Lf systems depends on the particular toxoid preparation.

In some embodiments of the invention a composition includes a ‘low dose’ of diphtheria toxoid. This means that the concentration of diphtheria toxoid in the composition is ≦8 Lf/ml e.g. <7, <6, <5, <4 <3, <2, <1 Lf/ml, etc. In a typical 0.5 ml unit dose volume, therefore, the amount of diphtheria toxoid is less than 4 Lf e.g. <3, <2, <1, <½ Lf, etc.

Diphtheria toxoid in the composition is preferably adsorbed (more preferably totally adsorbed) onto an aluminium salt, preferably onto an aluminium hydroxide adjuvant.

### Tetanus Toxoid

Tetanus is caused by Clostridium tetani, a Gram-positive, spore-forming bacillus. This organism expresses an endopeptidase (′tetanus toxin′), which can be treated to give a toxoid that is no longer toxic but that remains antigenic and is able to stimulate the production of specific anti-toxin antibodies after injection. Tetanus toxoids are disclosed in more detail in chapter 27 of reference 1. Preferred tetanus toxoids are those prepared by formaldehyde treatment. The tetanus toxoid can be obtained by growing C. tetani in growth medium (e.g. a Latham medium derived from bovine casein), followed by formaldehyde treatment, ultrafiltration and precipitation. The material may then be treated by a process comprising sterile filtration and/or dialysis.

Quantities of tetanus toxoid can be expressed in international units (IU). For example, NIBSC supplies the ‘Tetanus Toxoid Adsorbed Third International Standard 2000’ [12,13], which contains 469 IU per ampoule. As with diphtheria toxoid, the ‘LI’ unit is an alternative to the IU system. NIBSC supplies ‘The 1st International Reference Reagent for Tetanus Toxoid For Flocculation Test’ [14] which contains 1000 LF per ampoule. The concentration of diphtheria toxin in a composition can readily be determined using a flocculation assay by comparison with a reference material calibrated against such reference reagents.

In some embodiments of the invention a composition includes a ‘low dose’ of tetanus toxoid. This means that the concentration of tetanus toxoid in the composition is ≦3.5 Lf/ml e.g. <3, <2.5, <2, <1.5<1, <½ Lf/ml, etc. In a typical 0.5 ml unit dose volume, therefore, the amount of tetanus toxoid is less than 1.75 Lf e.g. <1.5, <1, <½, <¼Lf, etc.

Tetanus toxoid in the composition is preferably adsorbed (sometimes totally adsorbed) onto an aluminium salt, preferably onto an aluminium hydroxide adjuvant.

### Pertussis Toxoid

Bordetella pertussis causes whooping cough. Pertussis antigens in vaccines are either cellular (whole cell, in the form of inactivated B. pertussis cells; ‘wP’) or acellular (‘aP’). Preparation of cellular pertussis antigens is well documented (e.g. see chapter 21 of reference 1) e.g. it may be obtained by heat inactivation of phase I culture of B. pertussis. Where acellular antigens are used, one, two or (preferably) three of the following antigens are included: (1) detoxified pertussis toxin (pertussis toxoid, or TT′); (2) filamentous hemagglutinin (‘FHA’); (3) pertactin (also known as the ‘69 kiloDalton outer membrane protein’). These three antigens can be prepared by isolation from B. pertussis culture grown in modified Stainer-Scholte liquid medium. PT and FHA can be isolated from the fermentation broth (e.g. by adsorption on hydroxyapatite gel), whereas pertactin can be extracted from the cells by heat treatment and flocculation (e.g. using barium chloride). The antigens can be purified in successive chromatographic and/or precipitation steps. PT and FHA can be purified by hydrophobic chromatography, affinity chromatography and size exclusion chromatography. Pertactin can be purified by ion exchange chromatography, hydrophobic chromatography and size exclusion chromatography, or by IMAC. FHA and pertactin may be treated with formaldehyde prior to use according to the invention. PT is preferably detoxified by treatment with formaldehyde and/or glutaraldehyde. As an alternative to this chemical detoxification procedure the PT may be a mutant PT in which enzymatic activity has been reduced by mutagenesis [15] (e.g. the 9K/129G double mutant [16]), but detoxification by chemical treatment is preferred.

The invention can use a PT-containing wP antigen or, preferably, a PT-containing aP antigen. When using an aP antigen a composition of the invention will typically, in addition to the PT, include FHA and, optionally, pertactin. It can also optionally include fimbriae types 2 and 3.

Quantities of acellular pertussis antigens are typically expressed in micrograms. In some embodiments of the invention a composition includes a ‘low dose’ of pertussis toxoid. This means that the concentration of pertussis toxoid in the composition is ≦5 μg/ml e.g. <4, <3, <2.5, <2, <1 μg/ml, etc. In a typical 0.5 ml unit dose volume, therefore, the amount of pertussis toxoid is less than 2.5 μg e.g. <2, <1.5, <1, <0.5 μg, etc.

It is usual that each of pertussis toxoid, FHA and pertactin are present in a composition of the invention. These may be present at various ratios (by mass), such as PT:FHA:p69 ratios of 16:16:5 or 5:10:6 or 20:20:3 or 25:25:8 or 10:5:3. Each of these three antigens will generally be present at <60 μg/ml e.g. each in the range of 4-50 μg/ml. A total pertussis antigen concentration of <120 μg/ml is typical. It is usual to have a mass excess of FHA relative to pertactin when both are present. Pertussis toxoid in the composition is preferably adsorbed (sometimes totally adsorbed) onto an aluminium salt, preferably onto an aluminium hydroxide adjuvant. Any FHA can also be adsorbed to an aluminium hydroxide adjuvant. Any pertactin can be adsorbed to an aluminium phosphate adjuvant.

### Hib Conjugates

Haemophilus influenzae type b (‘Hib’) causes bacterial meningitis. Hib vaccines are typically based on the capsular saccharide antigen (e.g. chapter 14 of ref. 1), the preparation of which is well documented (e.g. references 17 to 26). The Hib saccharide is conjugated to a carrier protein in order to enhance its immunogenicity, especially in children. Typical carrier proteins are tetanus toxoid, diphtheria toxoid, the CRM197 derivative of diphtheria toxoid, H. influenzae protein D, and an outer membrane protein complex from serogroup B meningococcus. Tetanus toxoid is a preferred carrier, as used in the product commonly referred to as ‘PRP-T’. PRP-T can be made by activating a Hib capsular polysaccharide using cyanogen bromide, coupling the activated saccharide to an adipic acid linker (such as (1-ethyl-3-(3-dimethylaminopropyl) carbodiimide), typically the hydrochloride salt), and then reacting the linker-saccharide entity with a tetanus toxoid carrier protein. The saccharide moiety of the conjugate may comprise full-length polyribosylribitol phosphate (PRP) as prepared from Hib bacteria, and/or fragments of full-length PRP. Conjugates with a saccharide:protein ratio (w/w) of between 1:5 (i.e. excess protein) and 5:1 (i.e. excess saccharide) may be used e.g. ratios between 1:2 and 5:1 and ratios between 1:1.25 and 1:2.5. In preferred vaccines, however, the weight ratio of saccharide to carrier protein is between 1:2.5 and 1:3.5. In vaccines where tetanus toxoid is present both as an antigen and as a carrier protein then the weight ratio of saccharide to carrier protein in the conjugate may be between 1:0.3 and 1:2 [27]. Administration of the Hib conjugate preferably results in an anti-PRP antibody concentration of ≧0.15 μg/ml, and more preferably ≧1 μg/ml, and these are the standard response thresholds.

Quantities of Hib antigens are typically expressed in micrograms. For conjugate antigens this figure is based on the saccharide content of the conjugate. In some embodiments of the invention a composition includes a ‘low dose’ of a Hib conjugate. This means that the concentration of Hib saccharide in the composition is ≦5 μg/ml e.g. <4, <3, <2.5, <2, <1, etc. In a typical 0.5 ml unit dose volume, therefore, the amount of Hib is less than 2.5 μg e.g. <2, <1.5, <1, <0.5, etc.

Hib conjugate can be adsorbed onto an aluminium salt or can be unadsorbed.

### Hepatitis B Virus Surface Antigen

Hepatitis B virus (HBV) is one of the known agents which causes viral hepatitis. The HBV virion consists of an inner core surrounded by an outer protein coat or capsid, and the viral core contains the viral DNA genome. The major component of the capsid is a protein known as HBV surface antigen or, more commonly, ‘HBsAg’, which is typically a 226-amino acid polypeptide with a molecular weight of ˜24 kDa. All existing hepatitis B vaccines contain HBsAg, and when this antigen is administered to a normal vaccinee it stimulates the production of anti-HBsAg antibodies which protect against HBV infection.

For vaccine manufacture, HBsAg can be made in two ways. The first method involves purifying the antigen in particulate form from the plasma of chronic hepatitis B carriers, as large quantities of HBsAg are synthesized in the liver and released into the blood stream during an HBV infection. The second way involves expressing the protein by recombinant DNA methods. HBsAg for use with the method of the invention is recombinantly expressed in yeast cells. Suitable yeasts include Saccharomyces (such as S. cerevisiae) or Hanensula (such as H. polymorpha) hosts.

Unlike native HBsAg (i.e. as in the plasma-purified product), yeast-expressed HBsAg is generally non-glycosylated, and this is the most preferred form of HBsAg for use with the invention. Yeast-expressed HBsAg is highly immunogenic and can be prepared without the risk of blood product contamination.

The HBsAg will generally be in the form of substantially-spherical particles (average diameter of about 20 nm), including a lipid matrix comprising phospholipids. Yeast-expressed HBsAg particles may include phosphatidylinositol, which is not found in natural HBV virions. The particles may also include a non-toxic amount of LPS in order to stimulate the immune system [28]. The particles may retain non-ionic surfactant (e.g. polysorbate 20) if this was used during disruption of yeast [29].

A preferred method for HBsAg purification involves, after cell disruption: ultrafiltration; size exclusion chromatography; anion exchange chromatography; ultracentrifugation; desalting; and sterile filtration. Lysates may be precipitated after cell disruption (e.g. using a polyethylene glycol), leaving HBsAg in solution, ready for ultrafiltration.

After purification HBsAg may be subjected to dialysis (e.g. with cysteine), which can be used to remove any mercurial preservatives such as thimerosal that may have been used during HBsAg preparation [30]. Thimerosal-free preparation is preferred.

The HBsAg is preferably from HBV subtype adw2.

Quantities of HBsAg are typically expressed in micrograms. In some embodiments of the invention a composition includes a ‘low dose’ of HBsAg. This means that the concentration of HBsAg in the composition is ≦5 μg/ml e.g. <4, <3, <2.5, <2, <1, etc. In a typical 0.5 ml unit dose volume, therefore, the amount of HBsAg is less than 2.5 μg e.g. <2, <1.5, <1, <0.5, etc.

HBsAg can be adsorbed onto an aluminium salt (preferably adsorbed onto an aluminium phosphate adjuvant).

### Inactivated Poliovirus Antigen (IPV)

Poliomyelitis can be caused by one of three types of poliovirus. The three types are similar and cause identical symptoms, but they are antigenically very different and infection by one type does not protect against infection by others. As explained in chapter 24 of reference 1, it is therefore preferred to use three poliovirus antigens with the invention—poliovirus Type 1 (e.g. Mahoney strain), poliovirus Type 2 (e.g. MEF-1 strain), and poliovirus Type 3 (e.g. Saukett strain). As an alternative to these strains (“Salk” strains), Sabin strains of types 1 to 3 can be used e.g. as discussed in references 31 & 32. These strains can be more potent than the normal Salk strains.

Polioviruses may be grown in cell culture. A preferred culture uses a Vero cell line, which is a continuous cell line derived from monkey kidney. Vero cells can conveniently be cultured microcarriers. Culture of the Vero cells before and during viral infection may involve the use of bovine-derived material, such as calf serum, and of lactalbumin hydrolysate (e.g. obtained by enzymatic degradation of lactalbumin). Such bovine-derived material should be obtained from sources which are free from BSE or other TSEs.

After growth, virions may be purified using techniques such as ultrafiltration, diafiltration, and chromatography. Prior to administration to patients, polioviruses must be inactivated, and this can be achieved by treatment with formaldehyde before the viruses are used in the process of the invention.

The viruses are preferably grown, purified and inactivated individually, and are then combined to give a bulk mixture for use with the invention.

Quantities of inactivated poliovirus (IPV) are typically expressed in the ‘DU’ unit (the “D-antigen unit” [33]). In some embodiments of the invention a composition includes a ‘low dose’ of a poliovirus. For a Type 1 poliovirus this means that the concentration of the virus in the composition is ≦20 DU/ml e.g. <18, <16, <14, <12, <10, etc. For a Type 2 poliovirus this means that the concentration of the virus in the composition is ≦4 DU/ml e.g. <3, <2, <1, <0.5, etc. For a Type 3 poliovirus this means that the concentration of the virus in the composition is ≦16 DU/ml e.g. <14, <12, <10, <8, <6, etc. Where all three of Types 1, 2 and 3 poliovirus are present the three antigens can be present at a DU ratio of 5:1:4 respectively, or at any other suitable ratio e.g. a ratio of 15:32:45 when using Sabin strains [31]. A low dose of antigen from Sabin strains is particularly useful, with ≦10 DU type 1, ≦20 DU type 2, and ≦30 DU type 3 (per unit dose).

Polioviruses are preferably not adsorbed to any adjuvant before they are formulated, but after formulation they may become adsorbed onto aluminium salt(s) in the composition.

### Further Antigens

As well as including D, T, Pa, HBsAg, Hib and/or poliovirus antigens, immunogenic compositions of the invention may include antigens from further pathogens. For example, these antigens may be from N. meningitidis (one or more of serogroups A, B, C, W135 and/or Y) or S. pneumoniae.

### Meningococcal Saccharides

Where a composition includes a Neisseria meningitidis capsular saccharide conjugate there may be one or more than one such conjugate. Including 2, 3, or 4 of serogroups A, C, W135 and Y is typical e.g. A+C, A+W135, A+Y, C+W135, C+Y, W135+Y, A+C+W135, A+C+Y, A+W135+Y, A+C+W135+Y, etc. Components including saccharides from all four of serogroups A, C, W135 and Y are useful, as in the MENACTRA™ and MENVEO™ products. Where conjugates from more than one serogroup are included then they may be present at substantially equal masses e.g. the mass of each serogroup's saccharide is within ±10% of each other. A typical quantity per serogroup is between 1 μg and 20 μg e.g. between 2 and 10 μg per serogroup, or about 4 μg or about 5 μg or about 10 μg. As an alternative to a substantially equal ratio, a double mass of serogroup A saccharide may be used.

Administration of a conjugate preferably results in an increase in serum bactericidal assay (SBA) titre for the relevant serogroup of at least 4-fold, and preferably at least 8-fold. SBA titres can be measured using baby rabbit complement or human complement [34].

The capsular saccharide of serogroup A meningococcus is a homopolymer of (α1→6)-linked N-acetyl-D-mannosamine-1-phosphate, with partial O-acetylation in the C3 and C4 positions. Acetylation at the C-3 position can be 70-95%. Conditions used to purify the saccharide can result in de-O-acetylation (e.g. under basic conditions), but it is useful to retain OAc at this C-3 position. In some embodiments, at least 50% (e.g. at least 60%, 70%, 80%, 90%, 95% or more) of the mannosamine residues in a serogroup A saccharides are O-acetylated at the C-3 position. Acetyl groups can be replaced with blocking groups to prevent hydrolysis [35], and such modified saccharides are still serogroup A saccharides within the meaning of the invention.

The serogroup C capsular saccharide is a homopolymer of (α2→9)-linked sialic acid (N-acetyl neuraminic acid, or ‘NeuNAc’). The saccharide structure is written as →9)-Neu p NAc 7/8 OAc-(α2→. Most serogroup C strains have O-acetyl groups at C-7 and/or C-8 of the sialic acid residues, but about 15% of clinical isolates lack these O-acetyl groups [36,37]. The presence or absence of OAc groups generates unique epitopes, and the specificity of antibody binding to the saccharide may affect its bactericidal activity against O-acetylated (OAc) and de-O-acetylated (OAc+) strains [38-40]. Serogroup C saccharides used with the invention may be prepared from either OAc+ or OAc− strains. Licensed MenC conjugate vaccines include both OAc− (NEISVAC-C™) and OAc+(MENJUGATE™ & MENINGITEC™) saccharides. In some embodiments, strains for production of serogroup C conjugates are OAc+ strains, e.g. of serotype 16, serosubtype P1.7a,1, etc. Thus C:16:P1.7a,1 OAc+ strains may be used. OAc+ strains in serosubtype P1.1 are also useful, such as the C11 strain. Preferred MenC saccharides are taken from OAc+ strains, such as strain C11.

The serogroup W135 saccharide is a polymer of sialic acid-galactose disaccharide units. Like the serogroup C saccharide, it has variable O-acetylation, but at sialic acid 7 and 9 positions [41]. The structure is written as: →4)-D-Neup5Ac(7/9OAc)-α-(2→6)-D-Gal-α-(1→.

The serogroup Y saccharide is similar to the serogroup W135 saccharide, except that the disaccharide repeating unit includes glucose instead of galactose. Like serogroup W135, it has variable O-acetylation at sialic acid 7 and 9 positions [41]. The serogroup Y structure is written as: →4)-D-Neup5Ac(7/90Ac)-α-(2→6)-D-Glc-α-(1→.

The saccharides used according to the invention may be O-acetylated as described above (e.g. with the same O-acetylation pattern as seen in native capsular saccharides), or they may be partially or totally de-O-acetylated at one or more positions of the saccharide rings, or they may be hyper-O-acetylated relative to the native capsular saccharides. For example, reference 42 reports the use of serogroup Y saccharides that are more than 80% de-O-acetylated.

The saccharide moieties in meningococcal conjugates may comprise full-length saccharides as prepared from meningococci, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. One depolymerisation method involves the use of hydrogen peroxide [43]. Hydrogen peroxide is added to a saccharide (e.g. to give a final H₂O₂ concentration of 1%), and the mixture is then incubated (e.g. at about 55° C.) until a desired chain length reduction has been achieved. Another depolymerisation method involves acid hydrolysis [44]. Other depolymerisation methods are known in the art. The saccharides used to prepare conjugates for use according to the invention may be obtainable by any of these depolymerisation methods. Depolymerisation can be used in order to provide an optimum chain length for immunogenicity and/or to reduce chain length for physical manageability of the saccharides. In some embodiments, saccharides have the following range of average degrees of polymerisation (Dp): A=10-20; C=12-22; W135=15-25; Y=15-25. In terms of molecular weight, rather than Dp, useful ranges are, for all serogroups: <100 kDa; 5 kDa-75 kDa; 7 kDa-50 kDa; 8 kDa-35 kDa; 12 kDa-25 kDa; 15 kDa-22 kDa. In other embodiments, the average molecular weight for saccharides from each of meningococcal serogroups A, C, W135 and Y may be more than 50 kDa e.g. ≧75 kDa, ≧100 kDa, ≧110 kDa, ≧120 kDa, ≧130 kDa, etc. [45], and even up to 1500 kDa, in particular as determined by MALLS. For instance: a MenA saccharide may be in the range 50-500 kDa e.g. 60-80 kDa; a MenC saccharide may be in the range 100-210 kDa; a MenW135 saccharide may be in the range 60-190 kDa e.g. 120-140 kDa; and/or a MenY saccharide may be in the range 60-190 kDa e.g. 150-160 kDa.

If a component or composition includes both Hib and meningococcal conjugates then, in some embodiments, the mass of Hib saccharide can be substantially the same as the mass of a particular meningococcal serogroup saccharide. In some embodiments, the mass of Hib saccharide will be more than (e.g. at least 1.5×) the mass of a particular meningococcal serogroup saccharide. In some embodiments, the mass of Hib saccharide will be less than (e.g. at least 1.5× less) the mass of a particular meningococcal serogroup saccharide.

Where a composition includes saccharide from more than one meningococcal serogroup, there is an mean saccharide mass per serogroup. If substantially equal masses of each serogroup are used then the mean mass will be the same as each individual mass; where non-equal masses are used then the mean will differ e.g. with a 10:5:5:5 μg amount for a MenACWY mixture, the mean mass is 6.25 μg per serogroup. In some embodiments, the mass of Hib saccharide will be substantially the same as the mean mass of meningococcal saccharide per serogroup. In some embodiments, the mass of Hib saccharide will be more than (e.g. at least 1.5×) the mean mass of meningococcal saccharide per serogroup. In some embodiments, the mass of Hib saccharide will be less than (e.g. at least 1.5×) the mean mass of meningococcal saccharide per serogroup [46].

### Meningococcal Polypeptides

The capsular saccharide of Neisseria meningitidis serogroup B is not a useful vaccine immunogen and so polypeptide antigens can be used instead. For instance, the “universal vaccine for serogroup B meningococcus” reported by Novartis Vaccines in reference 47 can be used with the invention, or the BEXSERO product discussed in reference 48.

A composition of the invention can include a factor H binding protein (fHBP) antigen. The fHBP antigen has been characterised in detail. It has also been known as protein ‘741’ [SEQ IDs 2535 & 2536 in ref. 49], ‘NMB1870’, ‘GNA1870’ [refs. 50-52], ‘P2086’, ‘LP2086’ or ‘ORF2086’ [53-55]. It is naturally a lipoprotein and is expressed across all meningococcal serogroups. The fHBP antigen falls into three distinct variants [56] and it is preferred to include antigens for all variants.

A composition of the invention may include a Neisserial Heparin Binding Antigen (NHBA) [57]. This antigen was included in the published genome sequence for meningococcal serogroup B strain MC58 [58] as gene NMB2132.

A composition of the invention may include a NadA antigen. The NadA antigen was included in the published genome sequence for meningococcal serogroup B strain MC58 [58] as gene NMB1994.

A composition of the invention may include a NspA antigen. The NspA antigen was included in the published genome sequence for meningococcal serogroup B strain MC58 [58] as gene NMB0663.

A composition of the invention may include a NhhA antigen. The NhhA antigen was included in the published genome sequence for meningococcal serogroup B strain MC58 [58] as gene NMB0992.

A composition of the invention may include an App antigen. The App antigen was included in the published genome sequence for meningococcal serogroup B strain MC58 [58] as gene NMB1985.

A composition of the invention may include an Omp85 antigen. Omp85 was included in the published genome sequence for meningococcal serogroup B strain MC58 [58] as gene NMB0182.

A composition of the invention may include a meningococcal outer membrane vesicle.

### Pneumococcal Saccharides

Streptococcus pneumoniae causes bacterial meningitis and existing vaccines are based on capsular saccharides. Thus compositions of the invention can include at least one pneumococcal capsular saccharide conjugated to a carrier protein.

The invention can include capsular saccharide from one or more different pneumococcal serotypes. Where a composition includes saccharide antigens from more than one serotype, these are preferably prepared separately, conjugated separately, and then combined. Methods for purifying pneumococcal capsular saccharides are known in the art (e.g. see reference 59) and vaccines based on purified saccharides from 23 different serotypes have been known for many years. Improvements to these methods have also been described e.g. for serotype 3 as described in reference 60, or for serotypes 1, 4, 5, 6A, 6B, 7F and 19A as described in reference 61.

Pneumococcal capsular saccharide(s) will typically be selected from the following serotypes: 1, 2, 3, 4, 5, 6A, 6B, 7F, 8, 9N, 9V, 10A, 11A, 12F, 14, 15B, 17F, 18C, 19A, 19F, 20, 22F, 23F and/or 33F. Thus, in total, a composition may include a capsular saccharide from 1, 2, 3, 4, 5, 6, 7, 8, 9, 10, 11, 12, 13, 14, 15, 16, 17, 18, 19, 20, 21, 22, 23 or more different serotypes. Compositions which include at least serotype 6B saccharide are useful.

A useful combination of serotypes is a 7-valent combination e.g. including capsular saccharide from each of serotypes 4, 6B, 9V, 14, 18C, 19F, and 23F. Another useful combination is a 9-valent combination e.g. including capsular saccharide from each of serotypes 1, 4, 5, 6B, 9V, 14, 18C, 19F and 23F. Another useful combination is a 10-valent combination e.g. including capsular saccharide from each of serotypes 1, 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; or 22F and 15B. A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F; 6A and 19A, etc.

Thus a useful 13-valent combination includes capsular saccharide from serotypes 1, 3, 4, 5, 6A, 6B, 7F, 9V, 14, 18C, 19 (or 19A), 19F and 23F e.g. prepared as disclosed in references 62 to 65. One such combination includes serotype 6B saccharide at about 8 μg/ml and the other 12 saccharides at concentrations of about 4 μg/ml each. Another such combination includes serotype 6A and 6B saccharides at about 8 μg/ml each and the other 11 saccharides at about 4 μg/ml each.

Suitable carrier proteins for conjugates include bacterial toxins, such as diphtheria or tetanus toxins, or toxoids or mutants thereof. These are commonly used in conjugate vaccines. For example, the CRM197 diphtheria toxin mutant is useful [66]. Other suitable carrier proteins include synthetic peptides [67,68], heat shock proteins [69,70], pertussis proteins [71,72], cytokines [73], lymphokines [73], hormones [73], growth factors [73], artificial proteins comprising multiple human CD4⁺ T cell epitopes from various pathogen-derived antigens [74] such as N19 [75], protein D from H. influenzae [76-78], pneumolysin [79] or its non-toxic derivatives [80], pneumococcal surface protein PspA [81], iron-uptake proteins [82], toxin A or B from C. difficile [83], recombinant Pseudomonas aeruginosa exoprotein A (rEPA) [84], etc.

Particularly useful carrier proteins for pneumococcal conjugate vaccines are CRM197, tetanus toxoid, diphtheria toxoid and H. influenzae protein D. CRM197 is used in PREVNAR™. A 13-valent mixture may use CRM197 as the carrier protein for each of the 13 conjugates, and CRM197 may be present at about 55-60 μg/ml.

Where a composition includes conjugates from more than one pneumococcal serotype, it is possible to use the same carrier protein for each separate conjugate, or to use different carrier proteins. In both cases, though, a mixture of different conjugates will usually be formed by preparing each serotype conjugate separately, and then mixing them to form a mixture of separate conjugates. Reference 85 describes potential advantages when using different carrier proteins in multivalent pneumococcal conjugate vaccines, but the PREVNAR™ product successfully uses the same carrier for each of seven different serotypes.

A carrier protein may be covalently conjugated to a pneumococcal saccharide directly or via a linker. Various linkers are known. For example, attachment may be via a carbonyl, which may be formed by reaction of a free hydroxyl group of a modified saccharide with CDI [86,87] followed by reaction with a protein to form a carbamate linkage. Carbodiimide condensation can be used [88]. An adipic acid linker can be used, which may be formed by coupling a free—NH₂ group (e.g. introduced to a saccharide by amination) with adipic acid (using, for example, diimide activation), and then coupling a protein to the resulting saccharide-adipic acid intermediate[89,90]. Other linkers include β-propionamido [91], nitrophenyl-ethylamine [92], haloacyl halides [93], glycosidic linkages [94], 6-aminocaproic acid [95], N-succinimidyl-3-(2-pyridyldithio)-propionate (SPDP) [96], adipic acid dihydrazide ADH [97], C₄ to C₁₂ moieties [98], etc.

Conjugation via reductive amination can be used. The saccharide may first be oxidised with periodate to introduce an aldehyde group, which can then form a direct covalent linkage to a carrier protein via reductive amination e.g. to the 8-amino group of a lysine. If the saccharide includes multiple aldehyde groups per molecule then this linkage technique can lead to a cross-linked product, where multiple aldehydes react with multiple carrier amines. This cross-linking conjugation technique is particularly useful for at least pneumococcal serotypes 4, 6B, 9V, 14, 18C, 19F and 23F.

A pneumococcal saccharide may comprise a full-length intact saccharide as prepared from pneumococcus, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. Depolymerisation can be used in order to provide an optimum chain length for immunogenicity and/or to reduce chain length for physical manageability of the saccharides. Where more than one pneumococcal serotype is used then it is possible to use intact saccharides for each serotype, fragments for each serotype, or to use intact saccharides for some serotypes and fragments for other serotypes.

Where a composition includes saccharide from any of serotypes 4, 6B, 9V, 14, 19F and 23F, these saccharides are preferably intact. In contrast, where a composition includes saccharide from serotype 18C, this saccharide is preferably depolymerised.

A serotype 3 saccharide may also be depolymerised, For instance, a serotype 3 saccharide can be subjected to acid hydrolysis for depolymerisation [62] e.g. using acetic acid. The resulting fragments may then be oxidised for activation (e.g. periodate oxidation, maybe in the presence of bivalent cations e.g. with MgCl₂), conjugated to a carrier (e.g. CRM197) under reducing conditions (e.g. using sodium cyanoborohydride), and then (optionally) any unreacted aldehydes in the saccharide can be capped (e.g. using sodium borohydride) [62]. Conjugation may be performed on lyophilized material e.g. after co-lyophilizing activated saccharide and carrier.

A serotype 1 saccharide may be at least partially de-O-acetylated e.g. achieved by alkaline pH buffer treatment [63] such as by using a bicarbonate/carbonate buffer. Such (partially) de-O-acetylated saccharides can be oxidised for activation (e.g. periodate oxidation), conjugated to a carrier (e.g. CRM197) under reducing conditions (e.g. using sodium cyanoborohydride), and then (optionally) any unreacted aldehydes in the saccharide can be capped (e.g. using sodium borohydride) [63]. Conjugation may be performed on lyophilized material e.g. after co-lyophilizing activated saccharide and carrier.

A serotype 19A saccharide may be oxidised for activation (e.g. periodate oxidation), conjugated to a carrier (e.g. CRM197) in DMSO under reducing conditions, and then (optionally) any unreacted aldehydes in the saccharide can be capped (e.g. using sodium borohydride) [99]. Conjugation may be performed on lyophilized material e.g. after co-lyophilizing activated saccharide and carrier.

One or more pneumococcal capsular saccharide conjugates may be present in lyophilised form.

Pneumococcal conjugates can ideally elicit anticapsular antibodies that bind to the relevant saccharide e.g. elicit an anti-saccharide antibody level ≧0.20μg/mL [100]. The antibodies may be evaluated by enzyme immunoassay (EIA) and/or measurement of opsonophagocytic activity (OPA). The EIA method has been extensively validated and there is a link between antibody concentration and vaccine efficacy.

### Aluminium Salt Adjuvants

Compositions of the invention include an aluminium salt adjuvant. Aluminium salt adjuvants currently in use are typically referred to either as “aluminium hydroxide” or as “aluminium phosphate” adjuvants. These are names of convenience, however, as neither is a precise description of the actual chemical compound which is present (e.g. see chapter 9 of reference 101, and chapter 4 of reference 102). The invention can use any of the “hydroxide” or “phosphate” salts that useful as adjuvants. Aluminium salts which include hydroxide ions are the preferred insoluble salts for use with the present invention as these hydroxide ions can readily undergo ligand exchange for adsorption of antigen and/or TLR agonists. Thus preferred salts for adsorption of TLR4 agonists are aluminium hydroxide and/or aluminium hydroxyphosphate. These have surface hydroxyl moieties which can readily undergo ligand exchange with phosphorus-containing groups (e.g. phosphates, phosphonates) to provide stable adsorption. An aluminium hydroxide adjuvant is most preferred.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 101). The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants e.g. with needle-like particles with diameters about 2 nm. The PZC of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate. They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 0.99. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls (chapter 9 of ref. 101).

The PO₄/Al⁺⁺⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al⁺⁺⁺/ml. The aluminium phosphate will generally be particulate. Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The PZC of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

In solution both aluminium phosphate and hydroxide adjuvants tend to form stable porous aggregates 1-10 μm in diameter [103].

A composition can include a mixture of both an aluminium hydroxide and an aluminium phosphate, and components may be adsorbed to one or both of these salts.

An aluminium phosphate solution used to prepare a composition of the invention may contain a buffer (e.g. a phosphate or a histidine or a Tris buffer), but this is not always necessary. The aluminium phosphate solution is preferably sterile and pyrogen-free. The aluminium phosphate solution may include free aqueous phosphate ions e.g. present at a concentration between 1.0 and 20 mM, preferably between 5 and 15 mM, and more preferably about 10 mM. The aluminium phosphate solution may also comprise sodium chloride. The concentration of sodium chloride is preferably in the range of 0.1 to 100 mg/ml (e.g. 0.5-50 mg/ml, 1-20 mg/ml, 2-10 mg/ml) and is more preferably about 3±1 mg/ml. The presence of NaCl facilitates the correct measurement of pH prior to adsorption of antigens.

A composition of the invention ideally includes less than 0.85 mg Al⁺⁺⁺ per unit dose. In some embodiments of the invention a composition includes less than 0.5 mg Al⁺⁺⁺ per unit dose. The amount of Al⁺⁺⁺ can be lower than this e.g. <250 μg, <200 μg, <150 μg, <100 μg, <75 μg, <50 μg, <25 μg, <10 μg, etc.

In some embodiments of the invention a composition has an Al⁺⁺⁺ concentration below 1.7 mg/ml. The concentration of Al⁺⁺⁺ can be lower than this e.g. <1 mg/ml, <800 μg/ml, <600 μg/ml, <500 μg/ml, <400 μg/ml, <300 μg/ml, <250 μg/ml, <200 μg/ml, <150 μg/ml, <100 μg/ml, <75 μg/ml, <50 μg/ml, <20 μg/ml, etc.

Where compositions of the invention include an aluminium-based adjuvant, settling of components may occur during storage. The composition should therefore be shaken prior to administration to a patient. The shaken composition will be a turbid white suspension.

### Toll-Like Receptor 4 Agonists

Compositions of the invention include a TLR4 agonist, and most preferably an agonist of human TLR4. TLR4 is expressed by cells of the innate immune system, including conventional dendritic cells and macrophages [104]. Triggering via TLR4 induces a signalling cascade that utilizes both the MyD88- and TRIF-dependent pathways, leading to NF-κB and IRF3/7 activation, respectively. TLR4 activation typically induces robust IL-12p70 production and strongly enhances Thl-type cellular and humoral immune responses.

Various useful TLR4 agonists are known in the art, many of which are analogs of endotoxin or lipopolysaccharide (LPS). For instance, the TLR4 agonist can be:
(i) 3d-MPL (i.e. 3-O-deacylated monophosphoryl lipid A; also known as 3-de-O-acylated monophosphoryl lipid A or 3-O-desacyl-4′-monophosphoryl lipid A). This derivative of the monophosphoryl lipid A portion of endotoxin has a de-acylated position 3 of the reducing end of glucosamine. It has been prepared from a heptoseless mutant of Salmonella minnesota, and is chemically similar to lipid A but lacks an acid-labile phosphoryl group and a base-labile acyl group. Preparation of 3d-MPL was originally described in ref. 105, and the product has been manufactured and sold by Corixa Corporation. It is present in GSK's ‘AS04’ adjuvant. Further details can be found in references 106 to 109.
(ii) glucopyranosyl lipid A (GLA) [110] or its ammonium salt e.g.

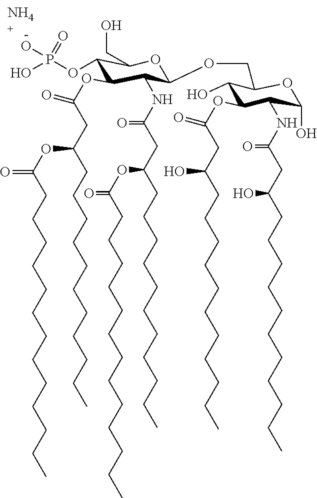

(iii) an aminoalkyl glucosaminide phosphate, such as RC-529 or CRX-524 [111-113]. RC-529 and CRX-524 have the following structure, differing by their R₂ groups:

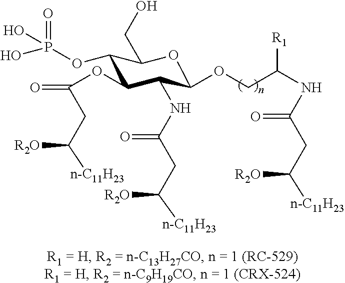

(iv) compounds containing lipids linked to a phosphate-containing acyclic backbone, such as the TLR4 antagonist E5564 [114,115]:

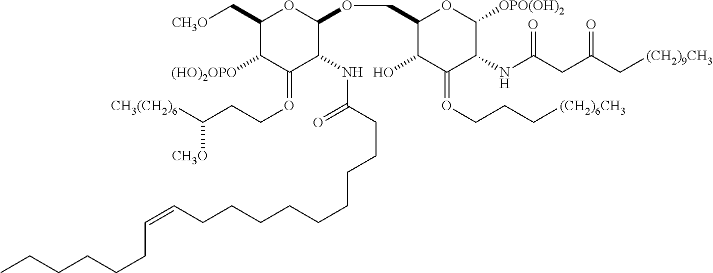

(v) A compound of formula I, II or III as defined in reference 116, or a salt thereof, such as compounds ‘ER 803058’, ‘ER 803732’, ‘ER 804053’, ‘ER 804058’, ‘ER 804059’, ‘ER 804442’, ‘ER 804680’, ‘ER 803022’, ‘ER 804764’ or ‘ER 804057’. ER 804057 is also known as E6020 and it has the following structure:

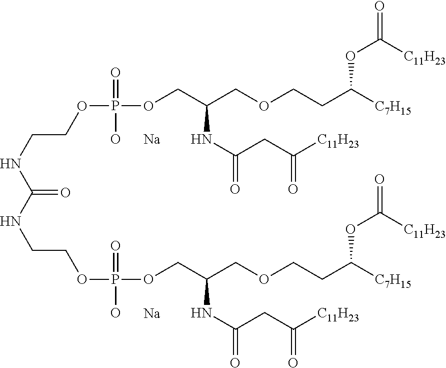

whereas ER 803022 has the following structure:

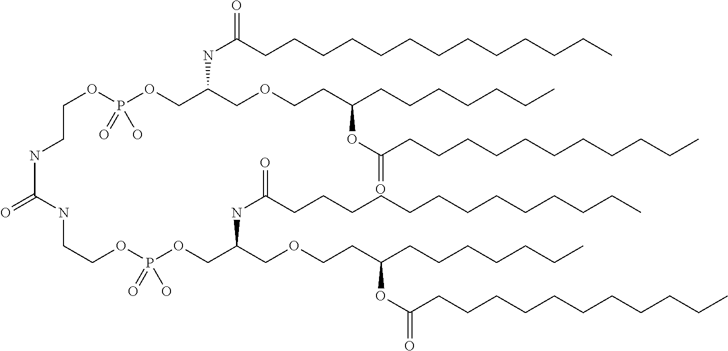 [SMILES: CCCCCCCCCCCCCC(=O)N[C@H](COCC[C@@H](CCCCCCC)OC(=O)CCCCCCCCCCC)COP(=O)(O)OCCNC(=O)NCCOP(=O)(O)OC[C@H](COCC[C@@H](CCCCCCC)OC(=O)CCCCCCCCCCC)NC(=O)CCCCCCCCCCCCC]

(vi) One of the polypeptide ligands disclosed in reference 117.
   Any of these TLR4 agonists can be used with the invention.

It is possible to adsorb TLR agonists to aluminium salts, thereby improving the immunopotentiating effect of the adjuvant [118]. This can lead to a better (stronger, or more quickly achieved) immune response and/or permits a reduction in the amount of aluminium in the composition while maintaining an equivalent adjuvant effect. A composition of the invention can therefore include an aluminium salt to which the TLR4 agonist is adsorbed. The agonist and the salt can form a stable adjuvant complex which retains (at least in part) the salt's ability to adsorb antigens.

TLR4 agonists with adsorptive properties typically include a phosphorus-containing moiety which can undergo ligand exchange with surface groups on an aluminium salt, and particularly with a salt having surface hydroxide groups. Thus a useful TLR4 agonist may include a phosphate, a phosphonate, a phosphinate, a phosphonite, a phosphinite, a phosphate, etc. Preferred TLR4 agonists include at least one phosphate group [118] e.g. the agonists (i) to (v) listed above.

The amount of TLR4 agonist in a unit dose will fall in a relatively broad range that can be determined through routine trials. An amount of between 1-1000 μg/dose can be used e.g. from 5-100 μg per dose or from 10-100 μg per dose, and ideally ≦300 μg per dose e.g. about 5 μg, 10 μg, 20 μg, 25 μg, 50 μg or 100 μg per dose. Thus the concentration of a TLR agonist in a composition of the invention may be from 2-2000 μg/ml e.g. from 10-200 μg/ml, or about 5, 10, 20, 40, 50, 100 or 200 μg/ml, and ideally ≦600 μg/ml.

In general, the weight ratio of TLR4 agonist to Al⁺⁺⁺ in a composition will be less than 5:1 e.g. less than 4:1, less than 3:1, less than 2:1, or less than 1:1. Thus, for example, with an Al⁺⁺⁺ concentration of 0.5 mg/ml the maximum concentration of TLR4 agonist would be 2.5 mg/ml. But higher or lower levels can be used. A lower mass of TLR4 agonist than of Al⁺⁺⁺ can be most typical e.g. per dose, 100 μg of TLR agonist with 0.2 mg Al⁺⁺⁺ etc. For instance, the Fendrix product includes 50 μg of 3d-MPL and 0.5 mg Al⁺⁺⁺ per dose.

It is preferred that at least 50% (by mass) of a TLR4 agonist in the composition is adsorbed to the aluminium salt e.g. ≧60%, ≧70%, ≧80%, ≧85%, ≧90%, ≧92%, ≧94%, ≧95%, ≧96%, ≧97%, ≧98%, ≧99%, or even 100%.

Where a composition of the invention includes a TLR4 agonist adsorbed to a metal salt, and also includes a buffer, it is preferred that the concentration of any phosphate ions in the buffer should be less than 50 mM (e.g. between 1-15 mM) as a high concentration of phosphate ions can cause desorption. Use of a histidine buffer is preferred.

### 3D-MPL

A preferred TLR4 agonist for use with the invention is 3d-MPL. This can be adsorbed to an aluminium phosphate adjuvant, to an aluminium hydroxide adjuvant, or to a mixture of both [119].

3d-MPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R^{2′}. The group attached to carbon 3′ has formula —O—CO—CH₂—CR³R^{3′}. A representative structure is:

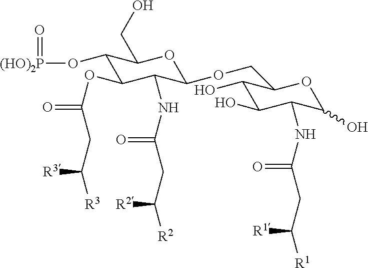

Groups R¹, R² and R³ are each independently —(CH₂)ₙ—CH₃. The value of n is preferably between 8 and 16, more preferably between 9 and 12, and is most preferably 10.

Groups R^{1′}, R^{2′} and R^{3′} can each independently be: (a)—H; (b)—OH; or (c)—O—CO—R⁴, where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are —H then the 3d-MPL has only 3 acyl chains (one on each of positions 2, 2′ and 3′). When only two of R^{1′}, R^{2′} and R^{3′} are —H then the 3d-MPL can have 4 acyl chains. When only one of R^{1′}, R^{2′} and R^{3′} is —H then the 3d-MPL can have 5 acyl chains. When none of R^{1′}, R^{2′} and R^{3′} is H then the 3d-MPL can have 6 acyl chains. The 3d-MPL used according to the invention can be a mixture of these forms, with from 3 to 6 acyl chains, but it is preferred to include 3d-MPL with 6 acyl chains in the mixture, and in particular to ensure that the 6 acyl chain form makes up at least 10% by weight of the total 3d-MPL e.g. ≧20%, ≧30%, ≧40%, ≧50% or more. 3d-MPL with 6 acyl chains has been found to be the most adjuvant-active form.

Thus the most preferred form of 3d-MPL for use with the invention is:

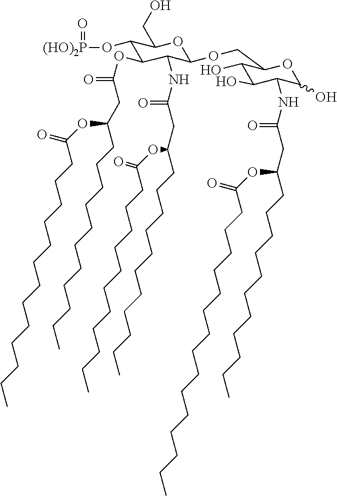

Where 3d-MPL is used in the form of a mixture then references to amounts or concentrations of 3d-MPL in compositions of the invention refer to the combined 3d-MPL species in the mixture.

Typical compositions include 3d-MPL at a concentration of between 25 μg/ml and 200 μg/ml e.g. in the range 50-150 μg/ml, 75-125 μg/ml, 90-110 μg/ml, or about 100 μg/ml. It is usual to administer between 25-75 μg of 3d-MPL per dose e.g. between 45-55 μg, or about 50 μg 3d-MPL per dose.

In aqueous conditions, 3d-MPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3d-MPL) are preferred for use according to the invention because of their superior activity [120]. Preferred particles have a mean diameter less than 150 nm, more preferably less than 120 nm, and can even have a mean diameter less than 100 nm. In most cases, however, the mean diameter will not be lower than 50 nm. Where 3d-MPL is adsorbed to an aluminum salt then it may not be possible to measure the 3D-MPL particle size directly, but particle size can be measured before adsorption takes place. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.

### Immunogenic Compositions

Compositions of the invention may comprise: (a) an antigenic component; and (b) a non-antigenic component. The antigenic component can comprise or consist of the antigens discussed above. The non-antigenic component can include carriers, adjuvants, excipients, buffers, etc., including the aluminium salt and the TLR4 agonist. These non-antigenic components may have various sources. For example, they may be present in one of the antigen or adjuvant materials that is used during manufacture or may be added separately from those components.

Preferred compositions of the invention include one or more pharmaceutical carrier(s) and/or excipient(s).

To control tonicity, it is preferred to include a physiological salt, such as a sodium salt. Sodium chloride (NaCl) is preferred, which may be present at between 1 and 20 mg/ml.

Compositions will generally have an osmolality of between 200 mOsm/kg and 400 mOsm/kg, preferably between 240-360 mOsm/kg, and will more preferably fall within the range of 280-320 mOsm/kg. Osmolality has previously been reported not to have an impact on pain caused by vaccination [121], but keeping osmolality in this range is nevertheless preferred.

Compositions of the invention may include one or more buffers. Typical buffers include: a phosphate buffer; a Tris buffer; a borate buffer; a succinate buffer; a histidine buffer; or a citrate buffer. Buffers will typically be included in the 5-20 mM range.

A composition of the invention can be substantially free from surfactants (prior to mixing with any emulsion adjuvant). In particular, the composition of the invention can be substantially free from polysorbate 80 e.g. it contains less than 0.1 μg/ml of polysorbate 80, and preferably contains no detectable polysorbate 80. Where a composition includes HBsAg, however, it will usually include polysorbate 20 e.g. if it was used during yeast disruption [29].

The pH of a composition of the invention will generally be between 6.0 and 7.5. A manufacturing process may therefore include a step of adjusting the pH of a composition prior to packaging. Aqueous compositions administered to a patient can have a pH of between 5.0 and 7.5, and more typically between 5.0 and 6.0 for optimum stability; where a diphtheria toxoid and/or tetanus toxoid is present, the pH is ideally between 6.0 and 7.0.

Compositions of the invention are preferably sterile.

Compositions of the invention are preferably non-pyrogenic e.g. containing <1 EU (endotoxin unit, a standard measure; 1 EU is equal to 0.2 ng FDA reference standard Endotoxin EC-2 ‘RSE’) per dose, and preferably <0.1 EU per dose.

Compositions of the invention are preferably gluten free.

Due to the adsorbed nature of antigens a vaccine product may be a suspension with a cloudy appearance. This appearance means that microbial contamination is not readily visible, and so the vaccine preferably contains an antimicrobial agent. This is particularly important when the vaccine is packaged in multidose containers. Preferred antimicrobials for inclusion are 2-phenoxyethanol and thimerosal. It is preferred, however, not to use mercurial preservatives (e.g. thimerosal) during the process of the invention. Thus, between 1 and all of the components used in the process may be substantially free from mercurial preservative. However, the presence of trace amounts may be unavoidable if a component was treated with such a preservative before being used in the invention. For safety, however, it is preferred that the final composition contains less than about 25 ng/ml mercury. More preferably, the final vaccine product contains no detectable thimerosal. This will generally be achieved by removing the mercurial preservative from an antigen preparation prior to its addition in the process of the invention or by avoiding the use of thimerosal during the preparation of the components used to make the composition. Mercury-free compositions are preferred.

Compositions of the invention will generally be in aqueous form.

During manufacture, dilution of components to give desired final concentrations will usually be performed with WFI (water for injection).

The invention can provide bulk material which is suitable for packaging into individual doses, which can then be distributed for administration to patients. Concentrations discussed above are typically concentrations in final packaged dose, and so concentrations in bulk vaccine may be higher (e.g. to be reduced to final concentrations by dilution).

Compositions of the invention are preferably administered to patients in 0.5 ml unit doses. References to 0.5 ml doses will be understood to include normal variance e.g. 0.5 ml±0.05 ml. For multidose situations, multiple dose amounts will be extracted and packaged together in a single container e.g. 5 ml for a 10-dose multidose container (or 5.5 ml with 10% overfill).

Residual material from individual antigenic components may also be present in trace amounts in the final vaccine produced by the process of the invention. For example, if formaldehyde is used to prepare the toxoids of diphtheria, tetanus and pertussis then the final vaccine product may retain trace amounts of formaldehyde (e.g. less than 10 μg/ml, preferably <5 μg/ml). Media or stabilizers may have been used during poliovirus preparation (e.g. Medium 199), and these may carry through to the final vaccine. Similarly, free amino acids (e.g. alanine, arginine, aspartate, cysteine and/or cystine, glutamate, glutamine, glycine, histidine, proline and/or hydroxyproline, isoleucine, leucine, lysine, methionine, phenylalanine, serine, threonine, tryptophan, tyrosine and/or valine), vitamins (e.g. choline, ascorbate, etc.), disodium phosphate, monopotassium phosphate, calcium, glucose, adenine sulfate, phenol red, sodium acetate, potassium chloride, etc. may be retained in the final vaccine at ≦100 μg/ml, preferably <10 μg/ml, each. Other components from antigen preparations, such as neomycin (e.g. neomycin sulfate, particularly from a poliovirus component), polymyxin B (e.g. polymyxin B sulfate, particularly from a poliovirus component), etc. may also be present at sub-nanogram amounts per dose. A further possible component of the final vaccine which originates in the antigen preparations arises from less-than-total purification of antigens. Small amounts of B. pertussis, C. cliphtheriae, C. tetani and S. cerevisiae proteins and/or genomic DNA may therefore be present. To minimize the amounts of these residual components, antigen preparations are preferably treated to remove them prior to the antigens being used with the invention.

Where a poliovirus component is used, it will generally have been grown on Vero cells. The final vaccine preferably contains less than 10 μg/ml, preferably ≦1 ng/ml e.g. ≦500 μg/ml or ≦50 μg/ml of Vero cell DNA e.g. less than 10 ng/ml of Vero cell DNA that is ≧50 base pairs long.

Compositions of the invention are presented for use in containers. Suitable containers include vials and disposable syringes (preferably sterile ones). Processes of the invention may comprise a step of packaging the vaccine into containers for use. Suitable containers include vials and disposable syringes (preferably sterile ones).

The invention also provides a delivery device (e.g. syringe, nebuliser, sprayer, inhaler, dermal patch, etc.) containing a pharmaceutical composition of the invention e.g. containing a unit dose. This device can be used to administer the composition to a vertebrate subject.

The invention also provides a sterile container (e.g. a vial) containing a pharmaceutical composition of the invention e.g. containing a unit dose.

The invention also provides a unit dose of a pharmaceutical composition of the invention.

The invention also provides a hermetically sealed container containing a pharmaceutical composition of the invention. Suitable containers include e.g. a vial.

Where a composition of the invention is presented in a vial, this is preferably made of a glass or plastic material. The vial is preferably sterilized before the composition is added to it. To avoid problems with latex-sensitive patients, vials may be sealed with a latex-free stopper. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. Preferred vials are made of colorless glass.

A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed.

Where the composition is packaged into a syringe, the syringe will not normally have a needle attached to it, although a separate needle may be supplied with the syringe for assembly and use. Safety needles are preferred. 1-inch 23-gauge, 1-inch 25-gauge and 5/8-inch 25-gauge needles are typical. Syringes may be provided with peel-off labels on which the lot number and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of butyl rubber. If the syringe and needle are packaged separately then the needle is preferably fitted with a butyl rubber shield. Grey butyl rubber is preferred. Preferred syringes are those marketed under the trade name “Tip-Lok”™.

Where a glass container (e.g. a syringe or a vial) is used, then it is preferred to use a container made from a borosilicate glass rather than from a soda lime glass.

After a composition is packaged into a container, the container can then be enclosed within a box for distribution e.g. inside a cardboard box, and the box will be labeled with details of the vaccine e.g. its trade name, a list of the antigens in the vaccine (e.g. ‘hepatitis B recombinant’, etc.), the presentation container (e.g. ‘Disposable Prefilled Tip-Lok Syringes’ or ‘10×0.5 ml Single-Dose Vials’), its dose (e.g. ‘each containing one 0.5 ml dose’), warnings (e.g. ‘For Adult Use Only’ or ‘For Pediatric Use Only’), an expiration date, an indication, a patent number, etc. Each box might contain more than one packaged vaccine e.g. five or ten packaged vaccines (particularly for vials).

The vaccine may be packaged together (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

The packaged vaccine is preferably stored at between 2° C. and 8° C. It should not be frozen.

Vaccines can be provided in full-liquid form (i.e. where all antigenic components are in aqueous solution or suspension) after manufacture, or they can be prepared in a form where the vaccine can be prepared extemporaneously at the time/point of use by mixing together two components. Such two-component embodiments include liquid/liquid mixing and liquid/solid mixing e.g. by mixing aqueous material with lyophilised material. For instance, in one embodiment a vaccine can be made by mixing: (a) a first component comprising aqueous antigens and/or adjuvant; and (b) a second component comprising lyophilized antigens. In another embodiment a vaccine can be made by mixing: (a) a first component comprising aqueous antigens and/or adjuvant; and (b) a second component comprising aqueous antigens. In another embodiment a vaccine can be made by mixing: (a) a first component comprising aqueous antigens; and (b) a second component comprising aqueous adjuvant. The two components are preferably in separate containers (e.g. vials and/or syringes), and the invention provides a kit comprising components (a) and (b).

Another useful liquid/lyophilised format comprises (a) an aqueous complex of an aluminium salt and a TLR4 agonist and (b) a lyophilised component including one or more antigens. A vaccine composition suitable for patient administration is obtained by mixing components (a) and (b). In some embodiments component (a) is antigen-free, such that all antigenic components in the final vaccine are derived from component (b); in other embodiments component (a) includes one or more antigen(s), such that the antigenic components in the final vaccine are derived from both components (a) and (b).

Thus the invention provides a kit for preparing a combination vaccine, comprising components (a) and (b) as noted above. The kit components are typically vials or syringes, and a single kit may contain both a vial and a syringe. The invention also provides a process for preparing such a kit, comprising the following steps: (i) preparing an aqueous component vaccine as described above; (ii) packaging said aqueous combination vaccine in a first container e.g. a syringe; (iii) preparing an antigen-containing component in lyophilised form; (iv) packaging said lyophilised antigen in a second container e.g. a vial; and (v) packaging the first container and second container together in a kit. The kit can then be distributed to physicians.

A liquid/lyophilised format is particularly useful for vaccines that include a conjugate component, particularly Hib and/or meningococcal and/or pneumococcal conjugates, as these may be more stable in lyophilized form. Thus conjugates may be lyophilised prior to their use with the invention.

Where a component is lyophilised it generally includes non-active components which were added prior to freeze-drying e.g. as stabilizers. Preferred stabilizers for inclusion are lactose, sucrose and mannitol, as well as mixtures thereof e.g. lactose/sucrose mixtures, sucrose/mannitol mixtures, etc. A final vaccine obtained by aqueous reconstitution of the lyophilised material may thus contain lactose and/or sucrose. It is preferred to use amorphous excipients and/or amorphous buffers when preparing lyophilised vaccines [122].

Compositions of the invention include diphtheria, tetanus and pertussis toxoids. In some embodiments the composition includes an excess of diphtheria toxoid relative to tetanus toxoid (as measured in Lf units). The excess is ideally at least 1.5:1 e.g. 5 Lf of diphtheria toxoid for every 2 Lf of tetanus toxoid (i.e. a 5:2 ratio). These embodiments are most useful in infants and children. In other embodiments, which are most useful in adolescents and adults (as a booster), the composition includes an excess of tetanus toxoid relative to diphtheria toxoid (as measured in Lf units). The excess is ideally at least 1.5:1 e.g. 2 Lf of tetanus toxoid for every 1 Lf of diphtheria toxoid (i.e. a 2:1 ratio). In other embodiments, equal amounts of diphtheria and tetanus toxoids are used (in Lf units). Where one of diphtheria or tetanus is present at an excess, the excess should ideally be at least 1.5-fold e.g. 2-fold or 2.5-fold, but the excess will not usually be more than 5-fold.

Some compositions of the invention include diphtheria, tetanus and pertussis toxoids, inactivated poliovirus for Types 1, 2 & 3, hepatitis B virus surface antigen and a Hib conjugate. The antigenic portion of these compositions may consist of the antigens in this list, or may further include antigens from additional pathogens (e.g. meningococcus). Thus the compositions can be used as vaccines themselves, or as components of further combination vaccines.

### Methods of Treatment, and Administration of the Vaccine

Compositions of the invention are suitable for administration to human patients, and the invention provides a method of raising an immune response in a patient, comprising the step of administering a composition of the invention to the patient.

The invention also provides a composition of the invention for use in medicine. The composition may be administered as variously described herein e.g. in some embodiments by giving an infant no more than two doses of a combination vaccine.

The invention also provides the use of a diphtheria toxoid, a tetanus toxoid, a pertussis toxoid, an aluminium salt adjuvant, and a TLR4 agonist in the manufacture of a medicament for raising an immune response in a patient. The medicament is ideally a composition as variously described elsewhere herein, and it can be administered as variously described herein.

The immune responses raised by these methods, uses and compositions are ideally protective, and immunogenic compositions of the invention are preferably vaccines, for use in the prevention of at least diphtheria, tetanus, and whooping cough. Depending on their antigen components the vaccines may also protect against bacterial meningitis, polio, hepatitis, etc.

In order to have full efficacy, a typical primary immunization schedule (particularly for a child) may involve administering more than one dose. For example, doses may be at: 0 & 6 months (time 0 being the first dose); at 0, 1, 2 & 6 months; at day 0, day 21 and then a third dose between 6 & 12 months; at 2, 4 & 6 months; at 3, 4 & 5 months; at 6, 10 & 14 weeks; at 2, 3 & 4 months; or at 0, 1, 2, 6 & 12 months.

Compositions can also be used as booster doses e.g. for children in the second year of life, for an adolescent, or for an adult.

Compositions of the invention can be administered by intramuscular injection e.g. into the arm or leg.

As mentioned above, a further aspect of the invention is an immunisation schedule for an infant (i.e. a child between birth and 1 year of age) in which only one or two DTP-containing compositions are administered. Thus, in some embodiments, the invention delivers fewer doses compared to the current normal 3-dose schedule, but without loss of immunoprotective effect. According to this aspect, no more than two doses of the vaccine are given to the infant i.e. the infant receives a single dose or two doses of the vaccine, but does not receive three (or more) doses. The infant may, though, receive a third (and maybe further) dose later in their life i.e. after their first birthday or after their second birthday. The one or two dose(s) is/are preferably given to the infant (i) between 1 and 5 months of age (ii) between 2 and 4 months of age (iii) between 3 and 5 months of age (iv) between 6 and 16 weeks of age or (v) between 0 and 3 months of age. For instance, two doses may be given at (i) 1 & 2 months of age (ii) 2 & 4 months of age (iii) 3 & 4 months of age (iv) 2 & 3 months of age (v) 0 and 1 months of age, etc.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where an antigen is described as being “adsorbed” to an adjuvant, it is preferred that at least 50% (by weight) of that antigen is adsorbed e.g. 50%, 60%, 70%, 80%, 90%, 95%, 98% or more. It is preferred that diphtheria toxoid and tetanus toxoid are both totally adsorbed i.e. none is detectable in supernatant. Total adsorption of HBsAg can be used.

Amounts of conjugates are generally given in terms of mass of saccharide (i.e. the dose of the conjugate (carrier+saccharide) as a whole is higher than the stated dose) in order to avoid variation due to choice of carrier.

Where a composition includes an aluminium salt adjuvant then preferably it does not also include an oil-in-water emulsion adjuvant. Conversely, where a composition includes an oil-in-water emulsion adjuvant then preferably it does not also include an aluminium salt adjuvant.

Phosphorous-containing groups employed with the invention may exist in a number of protonated and deprotonated forms depending on the pH of the surrounding environment, for example the pH of the solvent in which they are dissolved. Therefore, although a particular form may be illustrated herein, it is intended, unless otherwise mentioned, for these illustrations to merely be representative and not limiting to a specific protonated or deprotonated form. For example, in the case of a phosphate group, this has been illustrated as —OP(O)(OH)₂ but the definition includes the protonated forms—[OP(O)(OH₂)(OH)]⁺ and—[OP(O)(OH₂)₂]²⁺ that may exist in acidic conditions and the deprotonated forms—[OP(O)(OH)(O)]⁻ and [OP(O)(O)₂]²⁻ that may exist in basic conditions. The invention encompasses all such forms.

TLR4 agonists can exist as pharmaceutically acceptable salts. Thus, the compounds may be used in the form of their pharmaceutically acceptable salts i.e. physiologically or toxicologically tolerable salt (which includes, when appropriate, pharmaceutically acceptable base addition salts and pharmaceutically acceptable acid addition salts).

In the case of TLR agonists shown herein which may exist in tautomeric forms, the compound can be used in all such tautomeric forms.

Where a compound is administered to the body as part of a composition then that compound may alternatively be replaced by a suitable prodrug.

Where animal (and particularly bovine) materials are used in the culture of cells, they should be obtained from sources that are free from transmissible spongiform encephalopathies (TSEs), and in particular free from bovine spongiform encephalopathy (BSE).

### BRIEF DESCRIPTION OF THE DRAWINGS

FIG. 1 shows the % of FHA-specific memory B cells for the indicated treatment groups.

### MODES FOR CARRYING OUT THE INVENTION

Diphtheria toxoid, tetanus toxoid, pertussis toxoid, pertactin (p69), and filamentous hemagglutinin are prepared by conventional methods. These are combined in buffer at the following final concentrations (per ml) to make bulk vaccines ‘#1’ and ‘#2’:

| | Dt | Tt | Pt | p69 | FHA |
|---|---|---|---|---|---|
| #1 | 100 Lf | 40 Lf | 100 μg | 32 μg | 100 μg |
| #2 | 10 Lf | 20 Lf | 32 μg | 10 μg | 32 μg |

Vaccine #1 is intended for pediatric use, whereas vaccine #2 is intended for adolescent use.

Further bulk vaccines #3 and #4 are obtained in the same way, but they also include poliovirus types 1, 2 & 3 at 160, 32 and 128 DU/ml.

Further bulk vaccine #5 is obtained by adding HBsAg to vaccine #3 (40 μg/ml).

Other vaccines can be prepared from these four vaccines by dilution e.g. to reduce the dose while maintaining the ratio of antigens. Thus dilutions 2-fold, 2.5-fold, 3-fold, etc., can be made.

Vaccines #1, #2, #3, #4, and #5, or dilutions thereof, are combined with an adjuvant complex obtained by adsorbing an analog of monophosphoryl lipid either onto aluminium hydroxide or onto aluminium phosphate. This combination takes place at a 1:1 volume ratio, so the above antigen concentrations are halved in the final vaccine. The final Al⁺⁺⁺ concentration after mixing is 1 mg/ml.

Osmolarity and pH are measured (and, if necessary, adjusted) after combining the components in order to ensure physiological acceptability.

The integrity and immunogenicity of the combined antigens are tested, to check that none of the antigens shows an altered analytical profile after being formulated as combinations i.e. the antigens and adjuvants are physically compatible together.

The vaccines are then used to immunise test animals and immune responses are assessed. A typical dosage volume in mice is 0.1 ml (1/5 the human dosage volume).

### Booster Strength Vaccine

Three vaccines were tested, each containing (per 0.5 ml) 2 Lf of diphtheria toxoid, 5 Lf tetanus toxoid, and 16 μg acellular pertussis antigens (a mixture of purified PT-9K/129G, FHA and p69 pertactin). The excess of Tt relative to Dt provides dosing which is most useful in adolescents and adults.

The vaccines were (A) unadjuvanted (B) adjuvanted with 2 mg/ml aluminium hydroxide (‘Al—H’) or (C) adjuvanted with 2 mg/ml Al—H plus 100 μg/ml TLR-4 agonist. The TLR-4 agonist was a synthetic monophosphoryl lipid A and was adsorbed to the Al—H. All antigens were adsorbed to the Al—H in formulations (B) and (C).

For comparison the BOOSTRIX™ product was also tested. It contains (per 0.5 ml) 2.5 Lf of diphtheria toxoid, 5 Lf tetanus toxoid, and 18.5 μg acellular pertussis antigens (a mixture of purified PT, FHA and p69 pertactin), and it is adjuvanted with a mixture of aluminium phosphate and hydroxide salts. A mixture of buffer and Al—H was used as a negative control.

The four vaccines were administered to female Balb/C mice (6 weeks old) at 100 μl intramuscular doses on days 0, 21 and 35. Sera were tested 2 weeks after each dose.

Serum total IgG titers were measured against each antigen and were as follows (geometric means):

| | | | | (C) | | |
|---|---|---|---|---|---|---|
| | | (A) | (B) | AL—H + | | |
| Day | Ag | Unadj | Al—H | MPL | Boostrix | −ve control |
| 14 | Dt | 0.030 | 0.537 | 2.698 | 1.308 | 0.03 |
| | Tt | 0.064 | 1.448 | 8.700 | 2.48 | 0.03 |
| | PT | 7.255 | 5.549 | 10.27 | 1.629 | 1.52 |
| | FHA | 0.035 | 0.161 | 3.938 | 0.844 | 0.14 |
| | p69 | 1.365 | 5.730 | 44.65 | 14.46 | 1.67 |
| 35 | Dt | 0.030 | 45.36 | 95.18 | 85.55 | 0.03 |
| | Tt | 24.31 | 64.30 | 142.7 | 83.61 | 0.03 |
| | PT | 247.4 | 219.5 | 275.3 | 112.7 | 3.58 |
| | FHA | 7.433 | 43.94 | 97.00 | 37.58 | 0.07 |
| | p69 | 11.44 | 241.7 | 397.7 | 398.3 | 3.81 |
| 49 | Dt | 0.517 | 43.80 | 116.6 | 92.18 | 0.05 |
| | Tt | 40.21 | 54.20 | 213.9 | 99.09 | 0.03 |
| | PT | 353.6 | 297.6 | 509.2 | 162.8 | 5.23 |
| | FHA | 13.32 | 46.59 | 133.7 | 65.34 | 0.12 |
| | p69 | 36.91 | 256.9 | 474.6 | 403.6 | 1.95 |

Thus in all cases and at all time points (except for p69 at day 35) the highest titers in these 5 groups were seen in the mice which had received the antigens adjuvanted with adsorbed TLR4 agonist. Importantly, this improvement was seen even when compared to the licensed BOOSTRIX™ vaccine. Moreover, unlike Al—H alone or BOOSTRIX™, the adsorbed TLR4 agonist was able to improve anti-PT titers relative to the unadjuvanted group.

The TLR4 agonist also leads to more rapid responses. The second dose showed a clear increase in IgG responses for all antigens, but the improvements after the third dose were not so significant.

### FHA-Specific Memory B Cells

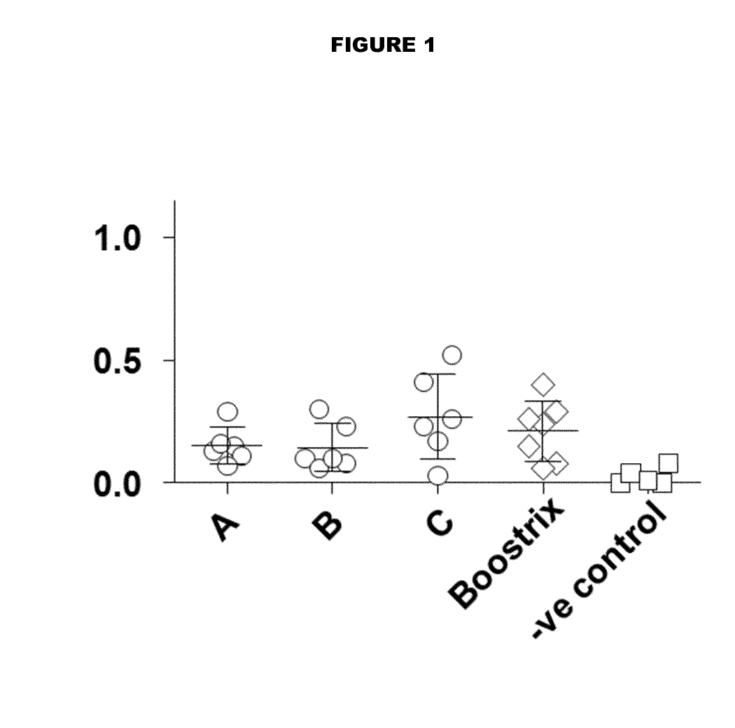

Four-to-five months after the third dose, FHA-specific memory B cells were measured in the immunised mice. The mice were sacrificed and their spleen cells were cultured in the presence of IL-2 and CpG for 5 days in order to expand all memory B cells. Spleen cells were then harvested and seeded in 96-well ELISPOT plates previously coated with either FHA antigen (10 mg/ml) or anti-mouse Ig. After overnight incubation, plates were washed to remove unattached spleen cells and both FHA-specific and total memory B cells were detected by biotinylated anti-mouse Ig and HRP-streptavidin. Colored spots, representing individual memory B cell, were counted with an ELISPOT reader instrument. The percentage of FHA-specific B cells compared to total B cells was then calculated for each sample, and FIG. 1 shows the results for each group. In these five groups the highest proportion is seen in group (C), with the TLR4 agonist.

It will be understood that the invention has been described by way of example only and modifications may be made whilst remaining within the scope and spirit of the invention.

TABLE A
antigen and Al⁺⁺⁺ content of various marketed vaccines (per unit dose)

| | D | | T | | Pa⁽¹⁾ | Hib⁽²⁾ | IPV⁽³⁾ | HBsAg | Vol | Al⁺⁺⁺ |
|---|---|---|---|---|---|---|---|---|---|---|
| Pediacel | 15 | Lf | 5 | Lf | 20/20/3 | 10 | 40/8/32 | — | 0.5 ml | 0.33 mg |
| Pediarix | 25 | Lf | 10 | Lf | 25/25/8 | — | 40/8/32 | 10 μg | 0.5 ml | ≦0.85 mg |
| Pentacel | 15 | Lf | 5 | Lf | 20/20/3 | 10 | 40/8/32 | — | 0.5 ml | 0.33 mg |
| Tritan^{x} HB | ≧30 | IU | ≧60 | IU | —⁽⁴⁾ | — | — | 10 μg | 0.5 ml | 0.63 mg |
| Quinvaxem | ≧30 | IU | ≧60 | IU | —⁽⁴⁾ | 10 | — | 10 μg | 0.5 ml | 0.3 mg |
| Hexavac | 30 | Lf | 10 | Lf | 25/25/— | 12 | 40/8/32 | 5 μg | 0.5 ml | 0.3 mg |
| Boostrix | 2.5 | Lf | 5 | Lf | 8/8/2.5 | — | — | — | 0.5 ml | ≦0.39 mg |
| Adacel | 2 | Lf | 5 | Lf | 2.5/5/3 | — | — | — | 0.5 ml | 0.33 mg |
| Daptacel | 15 | Lf | 5 | Lf | 10/5/3 | — | — | — | 0.5 ml | 0.33 mg |
| Pentavac | ≧30 | IU | ≧40 | IU | 25/25/— | 10 | 40/8/32 | — | 0.5 ml | 0.30 mg |
| SII QVac | 20-30 | Lf | 5-25 | Lf | —⁽⁴⁾ | — | — | ≧10 μg | 0.5 ml | ≦1.25 mg |
| TripVacHB | ≧30 | IU | ≧60 | IU | —⁽⁴⁾ | — | — | 10 μg | 0.5 ml | ≦1.25 mg |

Notes:
⁽¹⁾Pa dose shows amounts of pertussis toxoid, then FHA, then pertactin (μg). Pediacel's, Daptacel's and Adacel's Pa components also contain fimbriae types 2 and 3.
⁽²⁾Hib dose shows amount of PRP capsular saccharide (μg).
⁽³⁾IPV dose shows amounts of type 1, then type 2, then type 3 (measured in DU).
⁽⁴⁾Tritanrix-HepB, Quinvaxem, TripVac HB and SII Q-Vac include whole-cell pertussis antigens.

### REFERENCES

[1] Vaccines. (eds. Plotkin & Orenstein). 4th edition, 2004, ISBN: 0-7216-9688-0.
[2] Francois et al. (2005) Pediatr Infect Dis J24:953-61.
[3] Baylor et al. (2001) Vaccine 20 (Supplement 3):518-523
[4] Tamm et al. (2005) Vaccine 23:1715-19.
[5] WO2008/028956.
[6] National Institute for Biological Standards and Control; Potters Bar, UK. www.nibsc.ac.uk
[7] Sesardic et al. (2001) Biologicals 29:107-22.
[8] NIBSC code: 98/560.
[9] Module 1 of WHO's The immunological basis for immunization series (Galazka).
[10] NIBSC code: 69/017.
[11] NIBSC code: DIFT.
[12] Sesardic et al. (2002) Biologicals 30:49-68.
[13] NIBSC code: 98/552.
[14] NIBSC code: TEFT.
[15] Rappuoli et al. (1991) TIBTECH 9:232-238.
[16] Nencioni et al. (1991) Infect Immun. 59(2): 625-30.
[17] Ramsay et al. (2001) Lancet 357(9251):195-196.
[18] Lindberg (1999) Vaccine 17 Suppl 2:S28-36.
[19] Buttery & Moxon (2000) JR Coll Physicians Lond 34:163-168.
[20] Ahmad & Chapnick (1999) Infect Dis Clin North Am 13:113-133, vii.
[21] Goldblatt (1998) J. Med. Microbiol. 47:563-567.
[22] European patent 0477508.
[23] U.S. Pat. No. 5,306,492.
[24] WO98/42721.
[25] Conjugate Vaccines (eds. Cruse et al.) ISBN 3805549326, particularly vol. 10:48-114.
[26] Hermanson (1996) Bioconjugate Techniques ISBN: 0123423368 or 012342335X.
[27] WO96/40242.
[28] Vanlandschoot et al. (2005) J Gen Virol 86:323-31.
[29] WO2007/054820.
[30] WO03/066094.
[31] Liao et al. (2012) J Infect Dis. 205:237-43.
[32] Verdijk et al. (2011) Expert Rev Vaccines. 10:635-44.
[33] Module 6 of WHO's The immunological basis for immunization series (Robertson)
[34] W.H.O. Tech. Rep. Ser. 594:51, 1976.
[35] WO03/080678.
[36] Glode et al. (1979) J Infect Dis 139:52-56
[37] WO94/05325; U.S. Pat. No. 5,425,946.
[38] Arakere & Frasch (1991) Infect. Immun. 59:4349-4356.
[39] Michon et al. (2000) Dev. Biol. 103:151-160.
[40] Rubinstein & Stein (1998) J. Immunol. 141:4357-4362.
[41] WO2005/033148
[42] WO2005/000347.
[43] WO02/058737.
[44] WO03/007985.
[45] WO2007/000314.
[46] WO2007/000322.
[47] Giuliani et al. (2006) Proc Natl Acad Sci USA 103(29):10834-9.
[48] Bai et al. (2011) Expert Opin Biol Ther. 11:969-85.
[49] WO99/57280.
[50] Masignani et al. (2003) J Exp Med 197:789-799.
[51] Welsch et al. (2004) J Immunol 172:5605-15.
[52] Hou et al. (2005) J Infect Dis 192(4):580-90.
[53] WO03/063766.
[54] Fletcher et al. (2004) Infect Immun 72:2088-2100.
[55] Zhu et al. (2005) Infect Immun 73(10):6838-45.
[56] WO2004/048404
[57] Serruto et al. (2010) PNAS USA 107:3770-5.
[58] Tettelin et al. (2000) Science 287:1809-15.
[59] WHO Technical Report Series No. 927, 2005. Pages 64-98.
[60] US-2008/0102498.
[61] US-2006/0228381.
[62] US-2007/0231340.
[63] US-2007/0184072.
[64] US-2006/0228380.
[65] WO2008/143709.
[66] Research Disclosure, 453077 (January 2002)
[67] EP-A-0378881.
[68] EP-A-0427347.
[69] WO93/17712
[70] WO94/03208.
[71] WO98/58668.
[72] EP-A-0471177.
[73] WO91/01146
[74] Falugi et al. (2001) Eur J Immunol 31:3816-3824.
[75] Baraldo et al. (2004) Infect Immun 72(8):4884-7.
[76] EP-A-0594610.
[77] Ruan et al. (1990) J Immunol 145:3379-3384.
[78] WO00/56360.
[79] Kuo et al. (1995) Infect Immun 63:2706-13.
[80] Michon et al. (1998) Vaccine. 16:1732-41.
[81] WO02/091998.
[82] WO01/72337
[83] WO00/61761.
[84] WO00/33882
[85] WO2007/071707
[86] Bethell G. S. et al., J. Biol. Chem., 1979, 254, 2572-4
[87] Hearn M. T. W., J. Chromatogr., 1981, 218, 509-18
[88] WO2007/000343.
[89] Mol. Immunol., 1985, 22, 907-919
[90] EP-A-0208375
[91] WO00/10599
[92] Geyer et al., Med. Microbiol. Immunol, 165: 171-288 (1979).
[93] U.S. Pat. No. 4,057,685.
[94] U.S. Pat. Nos. 4,673,574; 4,761,283; 4,808,700.
[95] U.S. Pat. No. 4,459,286.
[96] U.S. Pat. No. 5,204,098
[97] U.S. Pat. No. 4,965,338
[98] U.S. Pat. No. 4,663,160.
[99] US-2007/0184071.
[100 ] Jodar et al. (2003) Vaccine 21:3265-72.
[101] Vaccine Design: The Subunit and Adjuvant Approach (eds. Powell & Newman) Plenum Press 1995 (ISBN 0-306-44867-X).
[102] Vaccine Adjuvants: Preparation Methods and Research Protocols (Volume 42 of Methods in Molecular Medicine series). ISBN: 1-59259-083-7. Ed. O'Hagan.
[103] Clausi et al. (2008) J Pharm Sci DOI 10.1002/jps.21390.
[104] Steinhagen et al. (2011) Vaccine 29:3341-55.
[105] GB-A-2220211.
[106] Myers et al. (1990) pages 145-156 of Cellular and molecular aspects of endotoxin reactions.
[107] Ulrich (2000) Chapter 16 (pages 273-282) of reference 102.
[108] Johnson et al. (1999) J Med Chem 42:4640-9.
[109] Baldrick et al. (2002) Regulatory Toxicol Pharmacol 35:398-413.
[110] Coler et al. (2011) PLoS ONE 6(1):e16333.
[111] Johnson et al. (1999) Bioorg Med Chem Lett 9:2273-2278.
[112] Evans et al. (2003) Expert Rev Vaccines 2:219-229.
[113] Bazin et al. (2006) Tetrahedron Lett 47:2087-92.
[114] Wong et al. (2003) J Clin Pharmacol 43(7):735-42.
[115] US2005/0215517.
[116] WO03/011223.
[117] WO2007/053455.
[118] WO2012/031140.
[119] Garcon et al. (2007) Expert Rev Vaccines 6:723-39.
[120] WO 94/21292.
[121] Nony et al. (2001) Vaccine 27:3645-51.
[122] WO01/41800.

1. A composition comprising a diphtheria toxoid, a tetanus toxoid, a pertussis toxoid, an aluminium salt adjuvant, and a TLR4 agonist.

2. The composition of claim 1, wherein the aluminium salt adjuvant is adsorbed to at least one component of the composition selected from the group consisting of the TLR4 agonist, the diphtheria toxoid, the tetanus toxoid, and the pertussis toxoid.

3. The composition of claim 1, wherein the composition comprises a concentration of Al⁺⁺⁺ of less than 0.4 mg/ml.

4. The composition of claim 1, wherein the composition comprises a low dose of each of the diphtheria toxoid, the tetanus toxoid, and the pertussis toxoid.

5. The composition of claim 1, further comprising at least one additional antigen.

6. The composition of claim 5, wherein the additional antigen is selected from the group consisting of conjugated Hib capsular saccharide, hepatitis B virus surface antigen, trivalent inactivated poliovirus, and conjugated meningococcal capsular saccharide.

7. The composition of claim 1, comprising ≦8 Lf/ml diphtheria toxoid.

8. The composition of claim 1, comprising ≦3.5 Lf/ml tetanus toxoid.

9. The composition of claim 1, comprising ≦5 μg/ml pertussis toxoid.

10. The composition of claim 1, further comprising ≦5 μg/ml Hib saccharide.

11. The composition of claim 1, further comprising ≦5 μg/ml HBsAg.

12. The composition of claim 1, further comprising at least one of (i)≦20 DU/ml type 1 poliovirus, (ii)≦4 DU/ml type 2 poliovirus and (iii)≦16 DU/ml type 3 poliovirus.

13. The composition of claim 1, wherein the aluminium salt adjuvant comprises an adjuvant selected from the group consisting of (i) an aluminium hydroxide adjuvant, (ii) an aluminium phosphate adjuvant, and (iii) a mixture of an aluminium hydroxide adjuvant, and an aluminium phosphate adjuvant.

14. The composition of claim 1, further comprising a conjugated capsular saccharide from one or more of meningococcal serogroups selected from the group of serogroups consisting of A, C, W135, and Y.

15. The composition of claim 1, further comprising a conjugated capsular saccharide from one or more of pneumococcal serotypes selected from the group of serogroups consisting of 1, 2, 3, 4, 5, 6A, 6B, 7F, 8, 9N, 9V, 10A, 11A, 12F, 14, 15B, 17F, 18C, 19A, 19F, 20, 22F, 23F, and 33F.

16. The composition of claim 1, further comprising one or more antigens selected from the group consisting of (i) a meningococcal factor H binding protein antigen, (ii) a Neisserial Heparin Binding Antigen, (iii) a meningococcal NhhA antigen, and (iv) a meningococcal outer membrane vesicle.

17. The composition of claim 1, wherein the TLR4 agonist comprises 3d-MPL.

18. A method of raising an immune response in a patient, comprising administering to the patient a composition of claim 1.

19. The composition of claim 1, wherein the composition is an immunogenic composition.
